(19) United States
(12) Patent Application Publication
(10) Pub. No.: US 2003/0164171 A1
(43) Pub. Date: Sep. 4, 2003

(54) Temperature alarm device for breathing apparatus

(76) Inventor: Sigurd Andersen, Sandefjord (NO)

Correspondence Address: KNOBBE MARTENS OLSON & BEAR LLP, 2040 MAIN STREET, FOURTEENTH FLOOR, IRVINE, CA, 92614, US

(21) Appl. No.: 10/149,271

(22) PCT Filed: Dec. 7, 2000
(86) PCT No.: PCT/NO00/00418

(30) Foreign Application Priority Data
Dec. 10, 1999 (NO) 19996101

(51) Int. Cl. A61M 16/00; A62B 7/00

(52) U.S. Cl. 128/204.26; 128/205.22; 128/205.24; 128/205.23

(57) ABSTRACT

A temperature alarm device for a breathing apparatus is described, comprising an air bottle (1) with a main valve (2) to which is connected a hose (3) for supply of air from the air bottle (1) via a reduction valve (4) to a breathing mask (5), wherein the temperature alarm device comprises a temperature controlled sound transmitter that emits a sound when the temperature of the surroundings exceeds a pre-defined temperature. A breathing apparatus comprising this temperature alarm device is also described.

The present invention regards a temperature alarm device for a breathing apparatus.

An ordinary breathing apparatus comprises an air bottle to be carried on the back, which feeds air to a mask covering the entire face, via a hose incorporating a reduction valve as breathing protection or a so-called “lung machine”. In order for the person using the breathing apparatus to be able to watch the pressure in the air bottle and thereby check that a sufficient amount of air remains in the bottle at all times, a pressure gauge is connected to the air bottle.

When using the breathing apparatus in smoky conditions, it may be difficult for the person using it to watch the pressure gauge. In order to warn the user when the pressure falls below a predetermined level, a whistle has also been connected to the air bottle, which opens when the pressure falls below the predetermined level, and sounds a warning.

Another danger of using a breathing apparatus in fire situations is that of the surrounding temperature increasing to a level at which the smoke in the room and/or the user's clothing is at risk from self-ignition. If the smoke and/or the clothes ignite, the user is in mortal danger. If there was a way of warning the person using the breathing apparatus that the temperature had exceeded a given value, this danger could be avoided, or at the very least reduced to a considerable degree.

Electronic equipment exists today, which may be used by persons using breathing apparatuses in order to measure temperatures. However this equipment is very expensive and requires a considerable amount of maintenance, such as changing the batteries etc., in order to ensure that it is operational at all times. Further, it does not include an alarm to warn the user of the temperature in cases where the task in hand may make it difficult for the user to watch the display.

It is an object of the present invention to provide an apparatus that warns the person using it when the surrounding temperature exceeds a danger level at which the user may be at risk from self-ignition. Furthermore, it is an object to make the apparatus simple and reasonable to construct and to ensure that it does not comprise batteries or other power sources that must be checked and changed out.

This object is achieved by a temperature alarm device for a breathing apparatus, comprising an air bottle with a main valve to which is connected a hose for supply of air from the air bottle via a reduction valve to the breathing mask, where the temperature alarm device comprises a temperature controlled sound transmitter that sounds a warning when the surrounding temperature exceeds a predefined temperature.

It is preferable that the sound transmitter comprise a whistle that is directly or indirectly connected to the air bottle, as well as a temperature controlled valve that opens above a certain temperature in order allow air from the air bottle to flow through the whistle, thus producing a sound.

It is further preferable for the temperature controlled valve to be a bimetal device.

It is also preferable for the temperature controlled valve to be a liquid based device.

According to a preferred embodiment, the temperature controlled valve is insulated in order to give a delayed tripping of the alarm.

Moreover, it is preferably that the sound transmitter and the temperature controlled valve be disposed at a good distance from the air bottle main valve.

The present temperature alarm device may if so desired be used in combination with a known electronic temperature gauge and form a supplement to this, as the present alarm device is unable to show the temperature development; it can only warn of excessive temperatures.

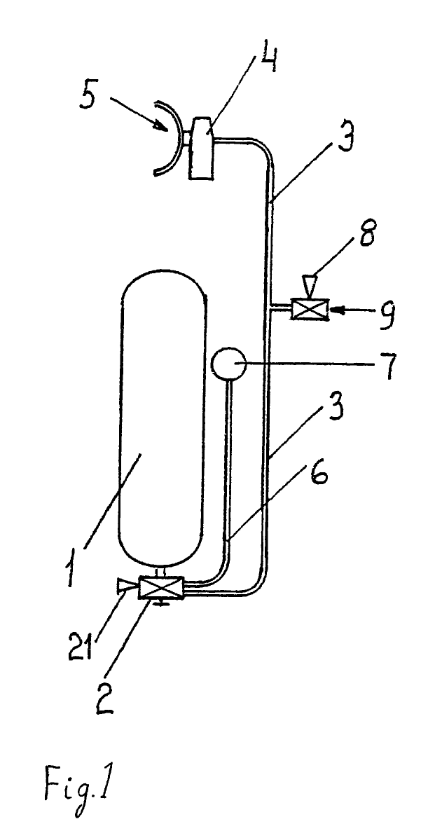

The invention will be described below with reference to the appended FIG. 1, which shows a schematic representation of the construction of a preferred breathing apparatus having a temperature alarm device according to the invention.

The appended Figure shows a preferred embodiment of the breathing apparatus according to the invention. The breathing apparatus comprises an air bottle 1 with a manually operated main valve 2, which apparatus supplies air to a mask 5 covering the entire face via a hose 3 with a reduction valve 4, also called a “lung machine”. The mask 5 covers the whole of the user's face, and is kept in place by straps that run behind the head. Further, a pressure gauge 7 with a gauge line 6 is normally provided in connection with the main valve 2, so as to allow the person using the breathing apparatus to read the pressure in the air bottle 1, and also a pressure controlled whistle that emits a loud, usually constant sound upon a reduced pressure in the air bottle 1.

In the Figure shown, the present temperature alarm device comprises a whistle 8 and a pressure controlled valve 9 connected to the hose 3. The temperature alarm device may be positioned in various ways. The important thing is for it to be supplied with pressurised air from the air bottle 1, either by means of the hose 3 or by means of another air supply. The temperature alarm device may thus be connected to a separate hose for supply of air, it may be connected directly to the air bottle, or it may be connected to the gauge line 6. However, it is preferable for the temperature alarm device to be placed at a point that, when the apparatus is in use, is located high up on the person using the breathing apparatus, e.g. on the back next to the air bottle 1. Preferably, it is located at a distance from the main valve, as the pressure drop across the main valve causes a reduction in temperature, and even riming in the area around the main valve 2, which can cause the temperature controlled valve 9 to be affected, and so not be tripped even if the surrounding temperature exceeds the predetermined temperature.

The temperature controlled valve 9 advantageously consists of a bimetal device or a liquid based device that opens for the pressurised air from the air supply for the temperature alarm device when the temperature exceeds a predetermined value. Alternatively the temperature controlled valve 9 may comprise a component having a defined melting point at which the valve is opened when the component melts.

When the temperature controlled valve 9 is open, pressurised air flows through the whistle 8, so that this emits a sound. Preferably, the whistle 8 emits a sound that differs significantly from the sound from whistle 21, to ensure that the person using the breathing apparatus knows with certainty which whistle is being sounded, and thereby which warning is being given. If the whistle 21 emits a constant sound, it is preferable for the whistle 8 to be of a type that emits an intermittent sound. Temperature controlled valves 9 of the bimetal type or the liquid based type normally return to the closed position upon cooling. This means that the valve 9 may be used again even if it has been opened. An alternative type of temperature controlled valve 9 may be of a type that opens when one of the components melts. The desired temperature for tripping the valve is selected by choosing a suitable material. A valve of this “melting” type will not return to its initial, closed position upon cooling, and will have to be replaced after having been tripped.

When a person uses breathing apparatus in a fire situation and the temperature of the room exceeds a set danger value above which the user may be at risk from flash fire, the temperature controlled valve 9 will open for supply of air from the air bottle to the whistle 8. The whistle 8 will then emit a sound signal, preferably an intermittent signal, so as to warn the user of the dangerous situation and allow him or her to withdraw from the danger zone and return to a safer area.

1. A temperature alarm device for a breathing apparatus comprising an air bottle (1) having a main valve (2) to which is connected a hose (3) for supply of air from the air bottle (1) via a reduction valve (4) to a breathing mask (5), where the temperature alarm device comprises a temperature controlled sound transmitter (8) that emits a sound when the temperature of the surroundings exceeds a predefined temperature, characterised in that the sound transmitter (8) comprises a whistle that is directly or indirectly connected to the air bottle (1), as well as a temperature controlled valve (9) that opens above a given temperature in order to allow air from the air bottle to flow through the whistle and thus produce a sound.

2. A temperature alarm device according to claim 1, characterised in that the temperature controlled valve (9) is a bimetal device.

3. A temperature alarm device according to claim 1, characterised in that the temperature controlled valve (9) is a liquid based device.

4. A temperature alarm device according to any of the preceding claim, characterised in that the temperature controlled valve (9) is insulated in order to delay the tripping of the sound emission.

5. A temperature alarm device according to any of the preceding claims, characterised in that the sound transmitter (8) and the temperature controlled valve (9) ane placed at a good distance from the main valve (2) of the air bottle.

6. A breathing apparatus comprising an air bottle (1) with a main valve (2) to which is connected a hose (3) for supply of air from the air bottle (1) via a reduction valve (4) to a breathing mask (5), the breathing apparatus comprises a temperature controlled sound transmitter (8) that emits a sound when the temperature of the surroundings exceeds a predefined temperature, characterised in that the sound transmitter (8) comprises a whistle directly or indirectly connected to the air bottle (1), as well as a temperature controlled valve (9) that opens above a given temperature in order to allow air from the air bottle to flow through the whistle and thus produce a sound.

7. A breathing apparatus according to claim 6, characterised in that the temperature controlled valve is a bimetal device.

8. A breathing apparatus according to claim 6, characterised in that the temperature controlled valve is a liquid based device.

9. A breathing apparatus device according to any of the claims 6 to 8, characterised in that the temperature controlled valve is insulated in order to delay the tripping of the sound emission.

10. A breathing apparatus device according to any of the claims 6 to 9, characterised in that the sound transmitter (8) and the temperature controlled valve are placed at a good distance from the main valve (2) of the air bottle.
